(19) United States
(12) Patent Application Publication
(10) Pub. No.: US 2005/0033340 A1
(43) Pub. Date: Feb. 10, 2005

(54) Concealed lancet cartridge for lancing device

(76) Inventors: Michael V. Lipoma, Villa Rica, GA (US); John C. Irwin, Woodstock, GA (US); Stephen J. Flynn, Peachtree City, GA (US)

Correspondence Address: GARDNER GROFF, P.C.;PAPER MILL VILLAGE, BUILDING 23, 600 VILLAGE TRACE, SUITE 300, MARIETTA, GA, 30067, US

(21) Appl. No.: 10/871,609

(22) Filed: Jun. 18, 2004

Related U.S. Application Data
(60) Provisional application No. 60/480,084, filed on Jun. 20, 2003.

(51) Int. Cl. A61B 17/14

(52) U.S. Cl. 606/181

(57) ABSTRACT

A method and apparatus for reducing user anxiety in the taking of a sample of blood. The apparatus comprises a lancing device that includes a housing and a disposable cartridge. The disposable cartridge is replaceably installed in the housing and includes a plurality of lancets within an outer shell which is effective for keeping the lancets hidden from view during loading and unloading of the disposable cartridge to and from the housing and during use of the lancing device.

### CROSS-REFERENCE TO RELATED APPLICATION

This application claims priority to U.S. Provisional Patent Application Ser. No. 60/480,084, filed Jun. 20, 2003, which is hereby incorporated herein by reference for all purposes.

### BACKGROUND OF THE INVENTION

The present invention relates generally to medical devices and procedures, and more particularly to lancing devices and methods that reduce the anxiety often associated with punctures of the skin for body fluid sampling.

### DESCRIPTION OF RELATED ART

Many medical procedures require puncturing of the skin, and sometimes underlying tissues, of a human or animal subject. For example, when administering a shot or drawing blood for testing using a hypodermic syringe, the needle of the syringe typically must puncture the subject's skin and enter underlying subcutaneous tissues. Needles are also used to collect blood from blood donors. Similarly, a sharp lancet tip is commonly used to puncture the subject's skin for sampling of blood or other body fluid, as for example in blood glucose monitoring by diabetics.

Many people are fearful of these procedures due to the pain and discomfort sometimes associated therewith and, as a result, avoid undertaking them even when the procedure is medically advisable. For example, many diabetics do not sample their blood sugar as often as is recommended, potentially leading to adverse health effects. Likewise, some persons avoid getting shots, such as flu shots, because they fear the pain of the needle-stick, potentially resulting in their contracting the flu or other ailment that could have been prevented had they taken the shot.

Thus, it has been found that a need exists for a method and apparatus for reducing the anxiety associated with skin-puncturing procedures. It is to the provision of a method and apparatus meeting this and other needs that the present invention is primarily directed.

### SUMMARY OF THE INVENTION

The present invention provides an apparatus and method for reducing the anxiety associated with skin-puncturing procedures. Generally described, the apparatus of the present invention includes one or more sharps, such as hypodermic needles or lancets enclosed within a shell that conceals the needles or lancets from the subject's view. Optionally, the shell can be operative to conceal the lancets from view at all relevant times, including during loading of the needles or lancets into the apparatus, during operation of the apparatus, and during removal of the needles or lancets after they have been used.

In example forms, the invention is a lancing device that includes a re-usable housing and a disposable cartridge. The disposable cartridge is provided for use in the housing and includes an internal lancet carrier including a plurality of lancets. The disposable cartridge preferably includes an outer shell which is effective for keeping the lancets hidden from view during loading/unloading of the disposable cartridge into the housing and during use of the lancing device.

Preferably, the housing is reusable and the cartridge is disposable. Also preferably, in a preferred form the carrier comprises a carousel style carrier. Optionally, the carousel carrier is advanced (indexed) one lancet at a time until all of the lancets have been used once. It is preferred that the lancing device include a mechanism for preventing the reuse of any individual lancet, for safety and hygiene. Also, an indicator can be provided for indicating how many lancets have been used or remain unused. Optionally, the lancing device can include a depth adjustment control. It is preferred that the depth adjustment control be operable and accessible without the necessity of opening the housing. In alternate embodiments, the cartridge comprises a plurality of lancets in a linear or rotary array, within a shell shielding the lancets themselves from view.

By keeping the lancets hidden from view, user anxiety can be greatly diminished. As is widely known, a great many people have an aversion to needles and other sharp objects. Even when the actual trauma induced by the lancing procedure is minimal, the perception of pain may be considerably increased due to the subject's anxiety leading up to the lancing procedure. In many subjects, this anxiety is increased upon observation of a lancet, which is at least subconsciously associated with the perceived pain. By keeping the lancet hidden from view at all times, anxiety can be reduced and people can remain more calm for skin puncturing procedures, such as while having a sample of blood taken or while taking a sample blood themselves. It is believed that by reducing user anxiety in this manner, users will be more likely to follow a prescribed regimen of regular sampling and monitoring.

This concept of hiding the lancets from view can be combined with other techniques to further reduce anxiety and discomfort in skin puncture. For example, the lancing device can be provided with a means for distracting or confusing the user or patient in a very slight manner so that the user or patient does not notice the prick of the lancets quite as distinctly. For example, an impact, vibration, acoustical signal or electrical stimulation can be imparted at or adjacent the puncture site prior to or simultaneously with the puncturing procedure. It has been found that nerve distraction or confusion generated in this manner lessens the sensation of pain resulting from the puncturing procedure in many subjects.

In one aspect, the invention is a lancing device including a cartridge having at least one lancet enclosed within a shell that obscures the at least one lancet from external view. The lancing device preferably also includes a housing for replaceably engaging the cartridge, the housing having a drive mechanism for propelling an active lancet from a retracted position through an extended position.

In another aspect, the invention is a disposable lancet cartridge for replaceable engagement within a housing. The cartridge preferably includes at least one lancet enclosed within a shell, each lancet being movable from a retracted position fully within the shell to an extended position wherein at least a tip portion of the lancet projects out of the shell. The shell preferably obscures the at least one lancet from external observation when in the retracted position.

In yet another aspect, the invention is a lancing device having a housing with first and second portions hingedly connected to one another, and a drive mechanism. The lancing device preferably also includes a cartridge having a plurality of lancets arranged in a generally circular array, the plurality of lancets being enclosed within a shell and being movable from a retracted position fully within the shell to an extended position wherein at least a tip portion of the lancet projects out of the shell. The shell preferably obscures the at least one lancet from external observation when in the retracted position.

These and other features and advantages of preferred forms of the present invention are described in greater detail herein with reference to preferred and example embodiments.

### BRIEF DESCRIPTION OF THE FIGURES

FIG. 1 is a perspective assembly view of a lancing device according to one exemplary embodiment of the invention.

FIG. 2 is a perspective view of the lancing device of FIG. 1, with the housing open and a lancet cartridge installed.

FIG. 3 is another perspective view of the lancing device of FIG. 1, with the housing closed.

FIGS. 4a-4d show a sequence of loading and operation of the lancing device of FIG. 1.

FIG. 5 shows a cross-sectional view of a lancing device according to the present invention.

FIG. 6 shows an assembly view of a lancet cartridge according to an example embodiment of the present invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

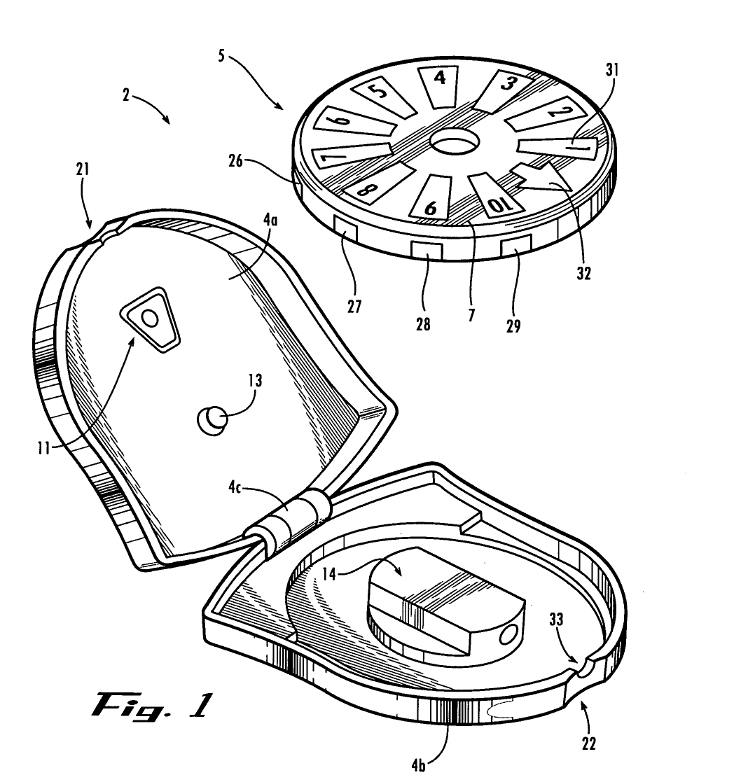

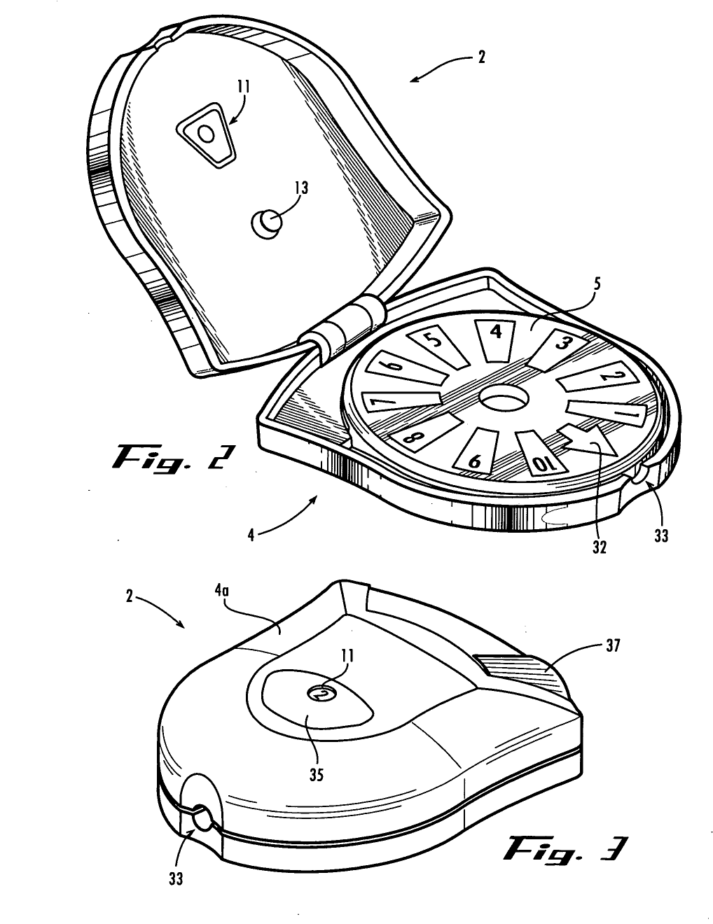

FIGS. 1-6 show a lancing device 2 for blood sample collection, according to an example embodiment of the invention. The lancing device 2 preferably comprises a clamshell housing 4, including an upper portion 4a, a lower portion 4b and a hinge 4c. In alternate embodiments, the housing is a substantially unitary enclosure with one or more openings for receiving and removing a cartridge, or is otherwise configured to removably house a cartridge. A lancet cartridge 5 is preferably removably mounted within the housing 4, and includes a lancet carrier 6 having at least one lancet 8, and preferably a plurality of individual lancets 8 and a cartridge cover 7 for concealing the individual lancets from view during loading/unloading of the cartridge and during use of the lancing device 2. As depicted in these figures, the carrier 6 and the cover 7 form a cartridge shell having the shape of a short, squat cylindrical disk, which is fitted atop and conceals the inner components of the cartridge 5, and which has a top face, a bottom face, and a sidewall extending between the top and bottom faces about the disk's circumference. In other embodiments, a separate lancet carrier 6 is enclosed within an outer cartridge shell.

The upper portion 4a of the housing 4 optionally includes a viewing window 11 to allow the user to determine how many lancets remain for use by reading indicia printed on the internal cartridge. The upper portion 4a preferably also includes a hub 13, generally centrally located on an underside of the upper portion 4a. This hub 13 cooperates with a recess in an upper surface of the cartridge 6 to rotationally secure the cartridge within the housing. The shape of the interior of the housing also helps in this regard inasmuch as it is sized and shaped to closely receive the cartridge. The lower portion 4b preferably comprises a circular recess or channel, concentric with the hub 13 in the upper housing portion when the housing is closed, for receiving the cartridge 5.

A drive mechanism 14 is preferably mounted to the lower housing portion, generally in the center of the recess or channel that receives the cartridge. The drive mechanism preferably comprises a spring-driven piston, cam-drive, solenoid, or other mechanical or electrical drive means for engaging sequential lancets of the cartridge and driving this “active” lancet from a retracted position within the cartridge, through an extended position wherein the sharp tip of the lancet extends outwardly of the device to penetrate the skin of a human or animal subject at a desired lancing site, and back to the retracted position with the tip of the used lancet shielded from external contact and view. The lancing device preferably also comprises an activation mechanism for manually triggering the release of the active lancet from the retracted position for blood sampling.

The upper and lower portions 4a, 4b of the housing 4 open and close about a hinge 4c, and when closed the housing has a forward end with a first radius of curvature, and a rear end that is generally flat or that has a substantially greater radius of curvature than the forward end. This housing geometry provides improved ergonomics, as the user can grasp the lancing device between the thumb and middle finger of one hand, and press the index finger of that hand against the rear end to better control applied pressure against the lancing site. The upper portion 4a and the lower portion 4b preferably have slight concave portions 21, 22 at the forward end of the housing, which meet when the two halves of the housing are closed together. The concave portions define a sampling location against which a finger or other body part is placed to allow it to be lanced in a controlled manner by the active lancet upon activation of the device. When the concave portions 21, 22 are adjacent to one another they define a lancet opening 33 through which the active lancet momentarily extends to lance the skin at a lancing site of the subject.

In preferred form, the cartridge is removable from the housing and disposable after use, and the housing 4 of the lancing device is preferably reusable. As best seen in FIG. 6, the cover 7 preferably includes a substantially rigid shell of plastic or other structural material, and defines one or more lancet passages 26-29 through the sidewall of shell, from which at least the sharp tip portions of the lancets emerge upon actuation of the lancing device. The lancet passage(s) align with the lancet aperture 33 in the housing 4 to permit the tip of the active lancet to extend outward of the housing to perform the lancing operation. The lancet passage(s) of the cartridge is/are preferably sufficiently small as to limit or obscure the lancet(s) within the cartridge from external observation by a user when cartridges are removed and replaced from the housing, and/or the lancet passage(s) is/are covered by foil, paper, or other penetrable material to block external view of the lancet(s). The individual lancets within the cartridge are preferably very small, low-mass lancets to minimize impact and user sensation. Because the lancet(s) are generally fully enclosed within the cartridge shell, a user preferably never sees or handles a lancet, a needle, or a lancet cap during use of the lancing device.

The cartridge 5 preferably also bears sequential numbers or other indicia 31 associated with each lancet, viewable through window 11 of the housing when the cartridge is loaded therein, to indicate how many lancets remain to be used or have been used. The cartridge 5 optionally also includes an alignment indicator 32, such as a marking, notch, projection, or other readily located feature, to help a user properly align the cartridge when installing/loading the cartridge into the housing 4 for use. The top of the cartridge cover preferably defines an opening or recess for rotational engagement with the hub 13 of the housing, and the bottom of the cartridge preferably comprises an opening for receiving the drive mechanism 14.

The lancing device 2 preferably also includes an advancing mechanism 37 for advancing (indexing) the internal disposable cartridge through sequential active lancets. In alternate embodiments, the advancing mechanism comprises a thumbwheel or gear rotationally mounted to the housing 4, a cam mechanism, an index wheel, a toggle arm, a motor drive, or other mechanism for cooperatively engaging the cartridge 5 or a portion thereof to sequentially advance lancets into and through the active lancet position. Preferably, the lancing device 2 also includes a one-way ratchet and pawl or other anti-reverse mechanism, allowing the cartridge to advance in one direction only, and thereby preventing the re-use of any previously used lancet. The device preferably also includes a stop or other lockout mechanism to allow the cartridge to be indexed through the sequence of lancets only once, to further maintain hygiene and safety.

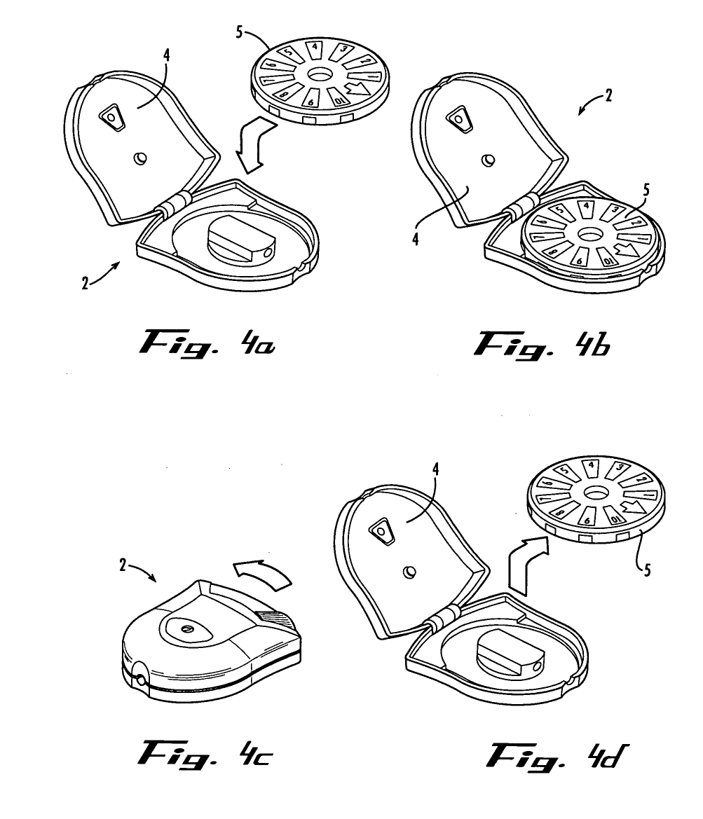

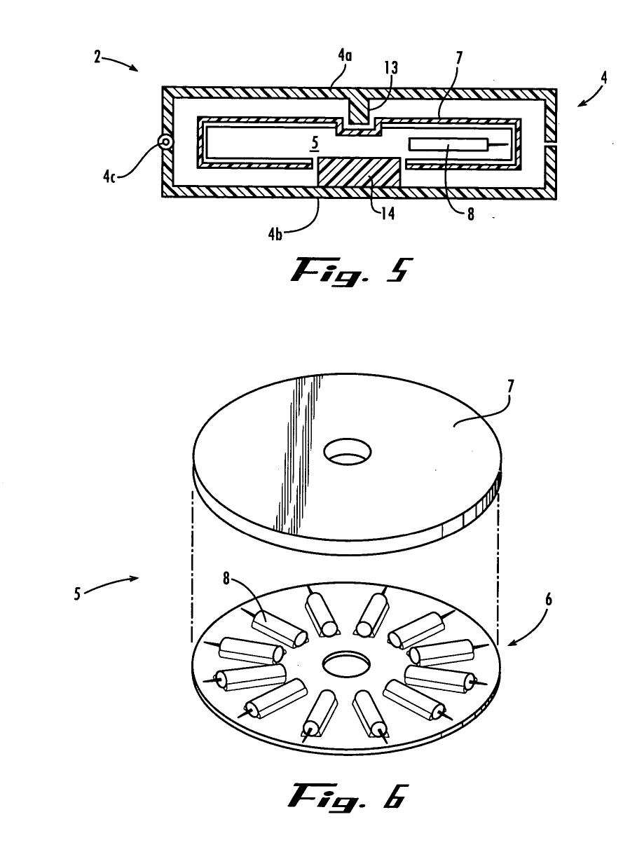

FIGS. 4a and 4b show the cartridge 5 being installed into the housing 4 for use. The alignment indicia 32 is aligned with the lancet aperture 33. The upper portion of the housing 4 is closed and the cartridge advanced once to ready the lancing device 2 for use. FIG. 4c shows the lancing device 2 closed and ready for use. A depth adjuster 35 is optionally provided to allow the user to vary the penetration depth of the lancets. The depth adjuster can, for example, take the form of a variable position stop or a movable member for varying the positioning of the lancing device at the lancing site. After actuating the device to carry out a lancing operation, the user advances the lancet cartridge to ready the next lancet for use, as indicated by the advancing direction arrow in FIG. 4c. This process is repeated for each lancing operation until all of the lancets in the cartridge have been used. After all of the lancets have been used, the user removes the used cartridge as shown in FIG. 4d, and disposes of it according to established disposal practice. There typically is no need to dispose of the cartridge in a “sharps” or other safety disposal container, since the cover 7 prevents unintentional contact with or contamination by the sharp lancet tips. Once the old cartridge is removed, a new cartridge can be placed in the housing and the housing cover closed again to ready the device for use once more.

The disposable cartridge arrangement of the present invention can be manufactured for different resellers with the housings being quite different in appearance and in some functionality, and with the cartridges having different appearances, but having common internal working mechanisms. In this manner, a manufacturerof lancet cartridges can use common internal workings (i.e., inner portions of the cartridge housed within the cartridge shell) in cartridges to be sold by multiple resellers, with the external shell of each reseller's cartridges having a unique appearance and/or structure. This allows for the production of proprietary (or apparently proprietary) lancing device cartridges to be sold to different resellers, and allowing the resellers to provide or prevent cross-compatibility of cartridges with different lancing devices as desired, while minimizing the cost of designing and manufacturing such because the internal workings of the cartridges can be common or universal to multiple cartridges.

Although the invention has been described primarily with respect to embodiments comprising lancets for blood sampling, other embodiments of the invention comprise one or more other type of sharps enclosed within a shell to obscure the sharps from external observation, and thereby reduce user anxiety. For example, one or more hypodermic needles, syringes, cannulas, trocars, and the like can be enclosed in a cartridge or shell in manners similar to the above-described embodiments. The present invention can be used to reduce anxiety in connection with any skin puncturing device or procedure, including for example lancing for sampling body fluids, injections, drawing blood, collecting blood and/or plasma from donors, insertion of scopes orother instruments in minimally-invasive surgery and the like, etc.; using a lancet, a syringe, a needle, a cannula, etc.

While the invention has been described with reference to preferred and example embodiments, it will be understood by those skilled in the art that a variety of modifications, additions and deletions are within the scope of the invention, as defined by the following claims.

1. A lancing device comprising:
a cartridge comprising at least one lancet enclosed within a shell that obscures the at least one lancet from external view; and
a housing for replaceably engaging the cartridge, said housing comprising a drive mechanism for propelling an active lancet from a retracted position through an extended position.

2. The lancing device of claim 1, wherein the cartridge comprises a plurality of lancets radially oriented in a generally circular array.

3. The lancing device of claim 1, wherein the shell of the cartridge defines at least one lancet passage for alignment with a lancet aperture in the housing to permit passage of a tip portion of the active lancet therethrough.

4. The lancing device of claim 3 wherein the at least one lancet passage is sufficiently small to obscure the lancets within the cartridge from external view.

5. The lancing device of claim 3 wherein the at least one lancet passage is covered with a penetrable material.

6. The lancing device of claim 1, wherein the cartridge comprises a carousel carrier carrying a plurality of lancets, and wherein the housing comprises an advancing mechanism to index sequential lancets of the plurality of lancets into engagement with the drive mechanism.

7. The lancing device of claim 1, wherein the cartridge comprises indicia showing how many lancets have been used or remain unused, and wherein the housing comprises a window for viewing said indicia through.

8. The lancing device of claim 1, wherein said housing comprises first and second portions hingedly connected to one another.

9. The lancing device of claim 1, wherein the shell of said cartridge comprises a generally circular disk having a top, a bottom, and a sidewall extending between the top and the bottom.

10. The lancing device of claim 9, wherein the top of the cartridge shell defines a recess for rotational engagement with a cooperating hub projecting from the housing.

11. The lancing device of claim 9, wherein the bottom of the cartridge defines an opening for receiving the drive mechanism.

12. The lancing device of claim 9, wherein the housing comprises a generally circular interior recess for receiving the cartridge therein.

13. The lancing device of claim 1, wherein the housing has a forward end with a first radius of curvature, and a rear end that is generally flat or that has a substantially greater radius of curvature than the forward end.

14. A disposable lancet cartridge for replaceable engagement within a housing, said cartridge comprising at least one lancet enclosed within a shell, each said at least one lancet being movable from a retracted position fully within the shell to an extended position wherein at least a tip portion of the lancet projects out of the shell, and wherein the shell obscures the at least one lancet from external observation when in the retracted position.

15. The cartridge of claim 14, further comprising at least one lancet passage through the shell through which at least a tip portion of the lancet projects in the extended position, and wherein the at least one lancet passage is sufficiently small as to obscure the at least one lancet from external observation by a user when cartridges are removed from and replaced into the housing.

16. The cartridge of claim 14, further comprising at least one lancet passage through the shell through which at least a tip portion of the lancet projects in the extended position, and wherein the at least one lancet passage is covered by a penetrable material to obscure external observation of the at least one lancet when cartridges are removed from and replaced into the housing.

17. The cartridge of claim 14, wherein the cartridge comprises a plurality of lancets radially oriented in a generally circular array.

18. The cartridge of claim 14, wherein the cartridge comprises a plurality of lancets and indicia showing how many lancets have been used or remain unused.

19. The cartridge of claim 14, wherein the shell comprises a generally circular disk having a top, a bottom, and a sidewall extending between the top and the bottom.

20. The cartridge of claim 19, wherein the top of the shell defines a recess for rotational engagement with a cooperating hub of the housing.

21. The cartridge of claim 19, wherein the bottom defines an opening for receiving a drive mechanism of the housing.

22. A lancing device comprising:
a housing comprising first and second portions hingedly connected to one another, and a drive mechanism; and
a cartridge comprising a plurality of lancets arranged in a generally circular array, said plurality of lancets being enclosed within a shell and being movable from a retracted position fully within the shell to an extended position wherein at least a tip portion of the lancet projects out of the shell, and wherein the shell obscures the at least one lancet from external observation when in the retracted position.
